(19) United States
(12) Patent Application Publication
(10) Pub. No.: US 2003/0064067 A1
(43) Pub. Date: Apr. 3, 2003

(54) Method for inducing differentiation and promoting proliferation of regulatory T cell

(75) Inventor: Junichi Masuyama, Tochigi (JP)

Correspondence Address: FOLEY AND LARDNER, SUITE 500, 3000 K STREET NW, WASHINGTON, DC, 20007, US

(73) Assignee: KIRIN BEER KABUSHIKI KAISHA

(21) Appl. No.: 10/122,432

(22) Filed: Apr. 16, 2002

(30) Foreign Application Priority Data
Oct. 1, 2001 (JP) 2001-305588

(51) Int. Cl. A61K 39/395

(52) U.S. Cl. 424/144.1; 424/141.1

(57) ABSTRACT

Provided is a method for inducing differentiation and/or promoting proliferation of regulatory T cells, which comprises allowing CD3 agonists and 4C8 antigen agonists to act on immunocytes expressing CD3 and 4C8 antigens.

This application claims a priority from Japanese Patent Application No. 2001-305588, the disclosure of which is incorporated herein by reference.

### BACKGROUND OF THE INVENTION

1. Field of the Invention

The present invention relates to a method for inducing differentiation and for promoting proliferation of regulatory T cells, and a pharmaceutical composition which is used in the method.

2. Description of Related Art

1. Regulatory T Cell

T cells compose a cell group which plays a central role in the immune system as a system of biophylaxis against various pathogens. T cells are largely classified into CD4⁺ helper T cells and CD8⁺ cytotoxic T cells. In particular, the former T cells can be classified, depending on cytokine-producing patterns at certain differentiation and maturation stages after stimulation with antigens, into, for instance, Th1 cells producing mainly IFN-γ and Th2 cells producing IL-4. Generally, the former and the latter T cells are deeply involved in biophylaxis as cellular immunity and as humoral immunity, respectively. The immune response is responsible for eliminating pathogens and acquiring resistance to infection based on a delicate balance resulting from functions of such T cells with varied characteristics. It is known that in the normal immune response, a mechanism works to eliminate foreign nonself antigens, but the mechanism does not eliminate autoantigens which make an organism because of the established immunologic tolerance. However, overresponse of the immune system against autoantigens causes a so-called autoimmune disease. As described above, immunological tolerance against autoantigens is not an absolute mechanism. The mechanisms by which various immunological torelances are induces are known in a T-cell level. One of such mechanisms, called central tolerance, eliminates autoreactive T cell clones in the thymus (Kisielow, P. et al., 1988. Nature. 333:742-746); and another mechanism, called peripheral tolerance, controls autoreactive T cells outside the thymus. Known as included in the latter mechanism are induction of cell death or of anergy against self-antigens (Rocha, B., and von Boehmer, H. 1991. Science. 251:1225-1228; Jenkins, M. K., and R. H. Schwartz. 1987. J. Exp. Med. 165:302-319.), and active suppression by regulatory T cells (Shevach, E. M. 2000. Annu. Rev. Immunol. 18:423449.). The regulatory T cell is a new, recently proposed concept of T cells, and is defined as having a suppressive action against other T cells. The immune response is operated based on a delicate balance. For example, the above Th1 cells and Th2 cells function antagonistically to their respective immune response, and it has become known that one acts as regulatory T cells on the other. However, verification of the presence of a cell population as regulatory T cells and property analysis thereof has been a point of considerable debate throughout the recent history of immunology. Such regulatory T cells have been studied in vitro or in vivo as cells capable of suppressing or regulating certain immune responses, so that the T cells have been reported as different cell populations according to the cell surface markers, the types of cytokines produced, suppressive and regulatory mechanisms and the like (Roncarolo, M. G., and M. K. Levings. 2000. Curr. Opinion. Immunol. 12:676-683.).

The most studied cell population among these regulatory T cells is a T cell population whose marker is CD4⁺CD25⁺ described below. This T cell population has been mainly studied in species of non-human organisms, such as mice and rats. The property analysis of the T cell population has been performed using as an index the fact that organ-specific autoimmune diseases (for example, thyroiditis, insulin-dependent diabetes mellitus, colitis) are induced by transferring T cells, from which certain T cells have been removed using expression of a particular cell surface molecule as an index, into T cell- and B cell-deficient SCID mice or rats (Sakaguchi, S., et al., 1985. J. Exp. Med. 161:72; Itoh, M., et al., 1999. J. Immunol. 162:5317-5326). Specifically, CD25⁺, RT6.1⁺ (expressed in most mature T cells in rat), CD5 highly positive, or CD45RB weakly positive (mice) or CD45RC weakly positive (rats) cells are removed from the CD4⁺ spleen cells of normal mice or rats, and then the remaining T cells are transferred to T cell- and B cell-deficient SCID mice or rats, thereby inducing organ-specific autoimmune diseases. To date, no such regulatory T cell-specific marker has been observed. That is, the above marker cannot be directly related to the function of regulatory T cells and it represents merely a state of cells being activated, that of cells being stimulated with antigens, or that of cells being immunological memory. However, the regulatory T cell population has been further analyzed using as an index the fact that the cell population is capable of suppressing autoimmune disease and autoimmune inflammation when a certain cell population is transferred, in addition to being capable of inducing organ-specific autoimmune disease in immunodeficient animals (Itoh, M., et al., 1999. J. Immunol. 162:5317-5326; Sakaguchi. S. et al., 1995. J. Immunol. 155:1151-1164; Asano, M. et al., 1996. J. Exp. Med. 184:387-396; Read, S. et al., 2000. J. Exp. Med. 192:295-302; Salomon, B. et al., 2000. Immunity. 12:431-440; Stephens, L. A., and D. Mason. 2000. J. Immunol. 165:3105-3110.). Therefore, it is now known that a CD4⁺CD25⁺T cell population is capable of using as a marker of the regulatory T cells, conventionally.

Though CD4⁺CD25⁺ regulatory T cells have been identified in mice and rats as described above, several groups just recently reported in 2001 the presence of similar cells in humans (Jonuleit, H. et al., 2001. J. Exp. Med. 193:1285-1294; Levings, M. K. et al., 2001. J. Exp. Med. 193:1295-1301; Dieckmann, D. et al., 2001. J. Exp. Med. 193:1303-1310; Taama, L. S. et al., 2001. Eur. J. Immunol. 31:1122-1131; Stephens, L. A. et al., 2001. Eur. J. Immunol. 31:1247-1245; Baecher-Allan, C. et al., 2001. J. Immunol. 167:1245-1253). The basis of these reports is that a cell population isolated from human peripheral blood, when expression of CD4 and CD25 known for mice is used as an index, has properties equivalent to those reported for mice, in terms of various cell surface markers, anergy of cells to stimulation for activation, types of cytokines produced, in vitro proliferation inhibitory function of normal T cells, the mechanism thereof, and the like. Specifically, CD4⁺CD25⁺T cells isolated from human peripheral blood express CD45R0⁺ memory T cell markers, and compared to CD4⁺CD25⁻ T cells, highly express activation markers such as HLA-DR. Further, CD4⁺CD25⁺ T cells constantly express CTLA-4 within the cells, and the expression of CTLA4 is enhanced by stimulation. Furthermore, some stimulations such as stimulation with anti-CD3 antibodies, stimulation with anti-CD3 antibodies and anti-CD28 antibodies, stimulation with allogeneic mature dendritic cells (allogeneic mature DC) do not cause CD4⁺CD25⁺ regulatory T cells to synthesize DNA and to produce cytokines. That is, CD4⁺CD25⁺ regulatory T cells are in an anergic state (anergy) following stimulation with antigens. Stimulation with cytokines, such as IL-2, IL-4, IL-15, in addition to that with anti-CD3 and anti-CD28 antibodies enhance the ability of CD4⁺CD25⁺ regulatory T cells to synthesize DNA, but the ability is not comparable to that of CD4⁺CD25⁻ T cells. When CD4⁺CD25⁻ T cells are stimulated with anti-CD3 antibodies or allogeneic mature DC in the presence of CD4⁺CD25⁺ regulatory T cells, in comparison with that in the absence of CD4⁺CD25⁺ regulatory T cells, proliferation inhibitory action is observed in a CD4⁺CD25⁺ regulatory T cell number-dependent manner. CD4⁺CD25⁺ regulatory T cells have ability to produce suppressor cytokines, such as IL-10 and TGFβ1, which is lower than that of mice. However, it has been reported that the proliferation inhibitory action against CD4⁺CD25⁻ T cells is not canceled by neutralizing antibodies against these cytokines and the inhibitory action requires direct intercellular contact between CD4⁺CD25⁻ T cells and CD4⁺CD25⁺ regulatory T cells. Though the presence of CD4⁺CD25⁺ regulatory T cells in a mice, rats and humans has been reported, and the property is being analyzed, detailed mechanisms of differentiation and suppressive action of these cells are still in the process of being elucidated, and no specific marker has been found so far.

Moreover, regulatory T cells which are induced in a mouse and a human by repeated stimulation with allogeneic antigens or allogeneic immature DC in the presence of IL-10 have been also reported (Groux, H. et al., 1997. Nature. 389:737-742; Jonuliet, H. et al., 2000. J. Exp. Med. 192:1213-1222). Unlike Th1 and Th2 cells, these cells called Tr1 cells are characterized by producing high levels of IL-10, moderate levels of TGF-β1, IFN-γ and IL-5, low levels of IL-2, and no IL-4. Similar to CD4⁺CD25⁺ regulatory T cells, Tr1 cells are anergic, and the T cell-suppressive mechanism can be partially explained by the IL-10 and TGFβ1 produced. However, whether Tr1 cells and CD4⁺CD25⁺ regulatory T cells are T cell subsets which are totally different from each other, or are the same cells but which differ in their differentiation activation stage remain almost unknown.

Using expression of regulatory T cell markers CD4 and CD25, known among mice and rats, as an index, CD4⁺CD25⁺ T cells have been isolated from human peripheral blood. Thus, the isolated T cells have been confirmed to share similar functions with other known cell surface markers of mice or rats, suggesting the presence of CD4⁺CD25⁺ regulatory T cells in humans.

These T cells are of a rare cell population which accounts for merely 5 to 10% of CD4⁺ T cells of peripheral blood, and are anergic to stimulation for activation and proliferation. In this case, cell proliferation can be promoted by stimulating with cytokines, such as IL-2, IL-4 and IL-15, in addition to anti-CD3 antibodies and anti-CD28 antibodies. However, this is not at a sufficient level for clinical applications, such as an application that involves increasing cell count and transferring the cells into a human.

Since regulatory T cells act suppressively on autoimmune disease, transplant rejection, graft versus host disease (GvHD) when transferred into an animal in an experiment (Hara, M. et al., 2001. J. Immunol. 166:3789-3796; Taylor, P. A. et al., 2001. J. Exp. Med. 193:1311-1317.), it is considered that regulatory T cells may be applied to cellular medicine using their immunosuppressive action to treat autoimmune disease, transplantation or the like. Development of a pharmaceutical composition which promotes proliferation of regulatory T cells, or development of a therapy which involves treating ex vivo peripheral blood or myeloma cells collected from patients or volunteers, allowing regulatory T cells to proliferate, and returning the cells into the bodies of patients, has been anticipated.

2. 4C8 Antigens

4C8 antigen is a membrane protein expressed on a part of cells of the immune system, and was originally found for the purpose of identifying molecules involving migration of human T cells to subendothelium after their adherence to vascular endothelial cells. Monoclonal antibodies obtained by immunizing mice with human T cells have been screened using inhibition of in vitro extravascular migration of T cells as an index, thereby obtaining monoclonal antibodies recognizing 4C8 antigens (Masuyama, J. et al., 1999. J. Exp. Med. 189:979-989; WO99/12972). A required process for T cells to migrate to peripheral tissues, such as an inflammatory focus, includes stable adherence of T cells to vascular endothelial cells via integrin molecules, and conformational change to a shape suitable for cell movement, followed by migration across the gap between vascular endothelial cells. It is known that not all the T cells adhered to vascular endothelial cells migrate to subendothelium, and that CD4⁺CD45RO⁺ CD26 highly positive activation memory T cells migrate selectively. Specifically, anti-4C8 monoclonal antibodies (mAb) do not inhibit the adherence of T cells to vascular endothelial cells, but specifically inhibit selective, transendothelial migration of T cell subsets. Hence, the 4C8 antigen is thought to be a functional molecule which is essential for such migration. When expression of the 4C8 antigens was verified with anti-4C8 mAb, the 4C8 antigens were found to be strongly expressed on CD3⁺ T cells and also expressed on B cells, NK cells, monocytes and eosinophils, but were not expressed on neutrophils or endothelial cells. Cross-inking with anti-4C8 mAb not only promotes actin polymerization of T cells and confers polarity to cell conformation, but also stimulates cell motility.

### SUMMARY OF THE INVENTION

The purpose of the present invention is to provide a method for inducing differentiation and promoting proliferation of regulatory T cells.

As a result of thorough studies to address the above problems, the present inventors have completed the present invention in the course of elucidating interaction between 4C8 antigens and anti-4C8 mAb by finding the above-mentioned novel function which is unpredictable from existing knowledge, that is, inhibiting migration of T cells. Specifically, we have found that under stimulation with CD3 agonists, action of anti-4C8 mAbs on immunocytes expressing 4C8 antigens causes costimulation, and thereby induces differentiation and promotes proliferation of regulatory T cells. The present invention is as described below.

1. A method for inducing differentiation and/or promoting proliferation of regulatory T cells, comprising allowing a CD3 agonist and a 4C8 antigen agonist to act on an immunocyte expressing CD3 and 4C8 antigens.

2. The method of 1, wherein the immunocyte expressing CD3 and 4C8 antigens is contained in peripheral blood, lymph nodes or thymic gland.

3. The method of 1, wherein the immunocyte expressing CD3 and 4C8 antigens is a T cell.

4. The method of 1, wherein the immunocyte expressing CD3 and 4C8 antigens is a peripheral blood mononuclear cell.

5. The method of 1, wherein the CD3 agonist is an anti-CD3 antibody.

6. The method of 1, wherein the 4C8 antigen agonist is an anti-4C8 antibody.

7. The method of 6, wherein the anti-4C8 antibody is a humanized antibody or a human antibody.

8. The method of 1, wherein the CD3 agonist and 4C8 antigen agonist act in vivo on an immunocyte expressing CD3 and 4C8 antigens.

9. The method of 1, wherein the CD3 agonist and 4C8 antigen agonist act ex vivo on an immunocyte expressing CD3 and 4C8 antigens.

10. The method of 1, wherein the regulatory T cell induced to differentiate expresses CD25 and CD152 and produces 1L-10.

11. The method of 1, wherein the regulatory T cell induced to differentiate suppresses cytokine production, proliferation and/or activation of other T cells.

12. A pharmaceutical composition for inducing differentiation and/or promoting proliferation of a regulatory T cell, comprising as an active ingredient a 4C8 antigen agonist.

13. The pharmaceutical composition of 12, wherein the 4C8 antigen agonist is an anti-4C8 antibody.

14. The pharmaceutical composition of 13, wherein the 4C8 antibody is a humanized antibody or a human antibody.

### BRIEF DESCRIPTION OF DRAWINGS

FIG. 1A shows the result of measuring the costimulation ability of anti-4C8 mAb to each T cell subset.

FIG. 1B shows the result of measuring by ELISA, using CD3+T cells, the concentration of IL-2 in the culture supernatant at 24 hours after stimulation.

FIG. 1C shows the result of comparing the expression of CD25 between the cells following 4C8 costimulation and those following CD28 costimulation 3 days after culturing. A thick solid line indicates CD25⁺ cells following 4C8 costimulation, and a thin solid line indicates CD25⁺ cells following CD28 costimulation. A broken line indicates a negative control group (antibodies with an isotype identical to that of anti-CD25 antibodies).

FIG. 1D shows the result of comparison between 4C8 costimulation and CD28 costimulation of viable cell count on days 2 to 5 after culturing, expressing cell count before culturing as 100%.

FIG. 2A shows the result of studying whether 4C8 costimulation induces polarization of T cells using production of IL-10 as an index.

FIG. 2B shows the result of studying whether 4C8 costimulation induces polarization of T cells using production of IL-2 as an index.

FIG. 3 shows the result of examining proliferative ability of T cells induced by 4C8 costimulation when stimulated with anti-CD3 mAb.

FIG. 4A shows the result of examining proliferation inhibitory effect of T cells induced by 4C8 costimulation on other T cells.

FIG. 4B shows the result of measuring, by ELISA, IL-2 in culture supernatant at 24 hours after co-culturing cells following 4C8 costimulation and freshly prepared T cells under stimulation with anti-CD3 antibodies. ● indicates production of IL-2 upon co-culturing with freshly prepared T cells, ∘ indicates production of IL-2 upon co-culturing with T cells following CD28 costimulation, and □ indicates production of IL-2 upon co-culturing with T cells following 4C8 costimulation.

FIG. 5A shows the result of analyzing by FACScan the proportion of cells expressing CD25 and CD152 on the cell surfaces when the cells on day 3 after 4C8 costimulation and CD28 costimulation were collected, washed, and then cultured without stimulation for a further 3 days or 6 days.

FIG. 5B shows the result of analyzing by FACScan the proportion of cells expressing CD4 on the cell surfaces and CD152 intracellularly when the cells on day 3 after 4C8 costimulation and CD28 costimulation were collected, washed, and then cultured without stimulation for a further 4 days.

### DETAILED DESCRIPTION OF THE INVENTION

A detailed description of the present invention will now be given.

In the present specification, the term “4C8 antigen agonist” means a substance which can induce either one or both of the following reactions (1) and (2) by intracellular signaling of a costimulation via 4C8 antigens, by acting on 4C8 antigens expressed on the surfaces of immunocytes: (1) differentiation of the cells into regulatory T cells, and (2) proliferation of the cells maintaining the properties of regulatory T cells. The 4C8 antigen agonists include natural ligands for 4C8 antigens, and antibodies against the 4C8 antigens. A specific example of the antibody is an anti-4C8 antibody produced by a hybridoma JM-1 (accession No. FERM BP-7757) that was deposited on Sep. 26, 2001 at the International Patent Organism Depositary (IPOD), National Institute of Advanced Industrial Science and Technology, Japan (1-1, Higashi 1-chome, Tsukuba-shi, Ibaraki, Japan). Antibodies which can be used in the present invention are not limited to the above anti-4C8 antibody. According to the report of Masuyama et al. (Masuyama, J. et al., 1999. J. Exp. Med. 189:979-989; WO99/12972), antibodies having the function as a 4C8 antigen agonist can be obtained by screening monoclonal antibodies collected from animals immunized with human T cells using as an index the inhibition of in vitro extravascular migration of T cells.

A desired antibody can be obtained efficiently using as antigens mononuclear cells, which is obtained by co-cultivation of a fraction of peripheral blood mononuclear cells collected from a human with human umbilical vein endothelial cells (HUVEC) cultured into the monolayer on a collagen gel, and followed by collection the cells which have migrated across a monolayer. Desired antibodies are more easily obtained by hybridoma screening in combination with screening which, by using anti4C8 antibodies produced by the hybridoma JM-1 as a competitive reagent, utilizes suppressed staining of T-cells by test antibodies as an index (that is, recognition of an epitope identical to that of an anti-4C8 antibody produced by the hybridoma JM-1). Further, antibodies used in the present invention can be easily obtained using a process for screening antibodies which recognize epitopes identical to those of anti-4C8 antibodies, in combination with a process for evaluating an ability to induce differentiation and proliferation of regulatory T cells described in Examples of the present invention. Further, for example, so-called humanized antibodies can be obtained as antibodies used in the present invention by transplanting the variable region of the anti-4C8 antibody produced by the hybridoma JM-1 into the framework region of a human antibody. Furthermore, human antibodies can be obtained as antibodies used in the present invention by using a mouse which retains a non-rearranged human antibody gene and produces a human antibody specific to an antigen upon immunization with the antigen (For example, Tomizuka et al., 2000. Proc. Natl. Acad. Sci. USA, 97:722).

The method of the present invention requires action of CD3 agonists in addition to that of 4C8 antigen agonists on immunocytes expressing 4C8 antigens. If a signal transduction system mediated by CD3 molecules, which is a major stimulus transduction pathway involved in differentiation of T cells, is taken to be a main stimulation pathway, a pathway mediated by 4C8 antigens is a so-called costimulation pathway. In the present specification, 4C8 costimulation refers that stimulation mediated by 4C8 antigens is given in addition to stimulation mediated by CD3 molecules. The term “CD3 agonist” in the present specification means a substance capable of inducing a reaction by acting on CD3 molecules expressed on the surfaces of immunocytes so as to cause intracellular signal transduction through CD3, and thereby promoting differentiation of the cells. Examples of the CD3 agonist include agonistic anti-CD3 antibodies, such as OKT3 (ATCC CRL-8001), UCHT1 (B. D. PharMingen), and HIT3a (B. D. PharMingen). Further, agonists for various T cell antigen receptors, in particular antibodies having agonistic action, can also be used as the CD3 agonist in the present invention, since these antibodies can cause the formation of complexes of T cell antigen receptors and CD3, resulting in intracellular signal transduction mediated by CD3. A specific example of the CD3 agonist is OT145 which is an antibody for a human T cell antigen receptor Vbeta6.7 (Posnett et al., 1986, Proc. Natl. Acad. Sci. USA., 83(20):7888-92).

The present invention provides a pharmaceutical composition which comprises as an active ingredient the 4C8 antigen agonist, and is used for inducing differentiation and/or promoting proliferation of regulatory T cells. The pharmaceutical composition of the present invention may be administered in vivo, or may be used for ex vivo treatment of immunocytes, or peripheral blood, lymph, lymph node cells and thymocytes containing immunocytes collected from patients or volunteers. The pharmaceutical composition of the present invention may be formulated by any known, standard method. Specifically, the pharmaceutical preparation may be produced by adding various therapeutically acceptable additives, such as a carrier, pH buffer, stabilizer and excipient. Such a preparation preferably contains a physiologically acceptable diluent or carrier. Examples of appropriate carriers include, but are not limited to, saline, phosphate buffered saline, phosphate buffered saline glucose solution and buffered saline. Alternatively, the 4C8 antigen agonist may also be in a re-constitutable freeze-dried form, to which the above buffering solution is added before use. Examples of the route of administering the above preparation include oral administration using a tablet, a capsule, granules, powder, syrup or the like; and parenteral administration using an injection, a drip, a suppository or the like.

The route and the dose of administration may be appropriately determined in the processes of preclinical and clinical tests, and may vary depending on, for example, symptom, age and body weight. Generally, in oral administration, daily dose for adults is approximately 0.01 mg to 1000 mg of the preparation, and it may be administered once or as divided dosage. Further, in parenteral administration, approximately 0.01 mg to 1000 mg of the preparation may be administered per single dose by subcutaneous, intramuscular or intravenous injection.

Diseases to be treated herein are those requiring treatment that achieves an immunosuppressive effect. Examples of such treatment include pre- and post-treatment for organ transplantation for the purpose of treating or preventing graft versus host disease (GvHD), and treatment or prevention of autoimmune diseases, such as rheumatism.

The present invention enables the employment of therapy that involves causing regulatory T cells to proliferate by ex vivo treatment of immunocytes, or peripheral blood, lymph, lymph node cells and thymocytes containing immunocytes collected from patients or volunteers, and returning the cells into the patients. A stem cell transplantation therapy that involves collecting peripheral blood or myeloma cells from a living body and returning the cells into a patient has already been conducted. Moreover, a cancer therapy that has been performed involves artificially treating dentritic cells, a kind of immunocyte, and returning the cells into the patient (M. Jefford, et al., The Lancet Oncology, 2: 343-353, June, 2001). Therapeutic or preventive effect can be achieved by adding 4C8 antigen agonists and CD3 agonists to the collected immunocytes to cause regulatory T cells to proliferate, and then returning the cells into the patients. Such a so-called ex vivo method can be said to almost truly reproduce at a field of therapy an experimental system that has been developed by basic research. In comparison with in vivo administration of a drug, which may be unable to produce an expected therapeutic effect due to influences such as internal absorption, metabolism or interference by unknown factors, the ex vivo method is a method which would produce a lower risk upon practical application.

### EXAMPLE

The present invention is further described in the Examples which illustrate specific embodiments and effects of the invention. However, the examples are provided for illustrative purposes only, and are not intended to limit the technical scope of the invention.

### Example 1

### Costimulation Ability of Anti-4C8 mAb for Activation of T Cells

To observe the costimulation ability of anti4C8 mAb for activation of T cells, the effect of anti4C8 mAb was examined using as indices proliferation abilities (DNA synthesis ability) of naive T cells (CD3⁺CD45RA⁺ cells) and memory T cells (CD3⁺CD45RO⁺ cells) derived from human peripheral blood, IL-2 production ability, expression of T cell activation markers (CD25), and increased number of viable cells, upon stimulation with immobilized anti-4C8 mAb in the presence of anti-CD3 antibodies at a concentration below the optimal level. The groups were compared with a control group stimulated with CD28 (anti-CD28 antibodies), which is widely recognized as a costimulation receptor for activating T cells.

Peripheral blood mononuclear cells were isolated by Ficoll-Hypaque density gradient centrifugation from heparinized human peripheral blood. When CD4⁺ T cells were used, the cells were prepared as follows. The fraction of T cell concentrate was prepared as a fraction not adsorbed to a nylon wool column. Analysis with a flowcytometer (FACScan, Becton-Dickinson, Mountain View, Calif.) revealed that the fraction contained 90% or more CD3⁺ cells. Flow cytometer analysis was performed by collecting 5×10³ events and using Cell Quest software (Becton-Dickinson). CD4⁺ T cells were prepared from the fraction by negative selection using a reagent according to the manufacturer's instructions (Stem Cell Technologies Inc., Vancouver, Canada). When human peripheral blood-derived T cell subsets were used, these cells were prepared as follows. CD3⁺CD45RA⁺ cells and CD3⁺, CD45RO⁺ cells were respectively prepared from peripheral blood mononuclear cells using a Cellect™ plus Human CD3⁺CD45RO⁻/CD3⁺CD45RA⁻ kit (Cytovax Biotechnologies Inc., Edmonton, Canada). The purity of each cell fraction ranged from 91% to 96% according to the results of flowcytometer analysis (FACScan, Becton-Dickinson, Mountain View, Calif.).

The costimulation ability of the anti-4C8 mAb for each T cell subset was measured by the following method. 0.1 μg/ml anti-CD3 mAb (OKT3, provided by Dr. S. Kashiwagi, Japan Immunoresearch Laboratories Co., Ltd, Gunma, Japan)-containing PBS (100 μl) was immobilized by adding it onto a 96-well flat bottom microtiter plate (Falcon, Becton-Dickinson), followed by incubation at 4C.° for 24 hours. Then, purified anti-4C8 mAb (10 μg/ml: 100 μl) was immobilized to the plate, similarly. After washing twice with PBS, 1% BSA/PBS (100 μl) was added to the plate, the plate was incubated at 37C.° for 1 hour, and then assayed. Anti-CD28 mAb (clone CD28.2, PharMingen, San Diego, Calif.) was added at a final concentration of 5 μg/ml upon the start of assay culture. The cells were suspended in RPMI 1640 (Sigma)/10% FCS/penicillin (100 U/ml)/streptomycin (100 μg/ml)/2 mM glutamine medium, the suspension was added to 2×10⁵ cells/well/200 μl, and then assayed. The cells were cultured at 37° C. for 3 days in the presence of 5% CO₂. At 8 to 16 hours before collecting cells, 0.2 μCi of ³H-thymidine (New England Nuclear, Boston, Mass.) was added, and then culturing was continued. The cells were collected on a glass filter, and then ³H-thymidine incorporated into the cells was measured using a liquid scintillation counter. Every 3 wells/group were assayed, and mean values and SD values were calculated.

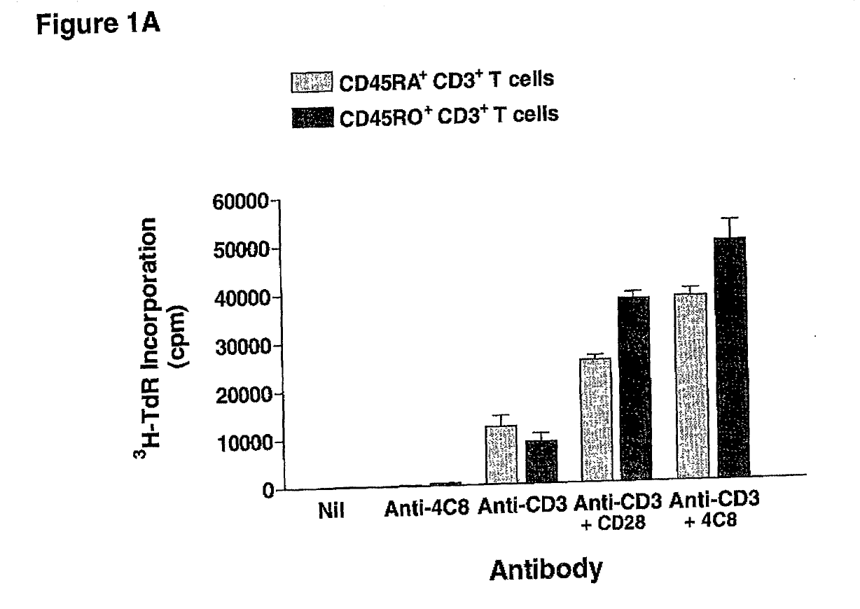

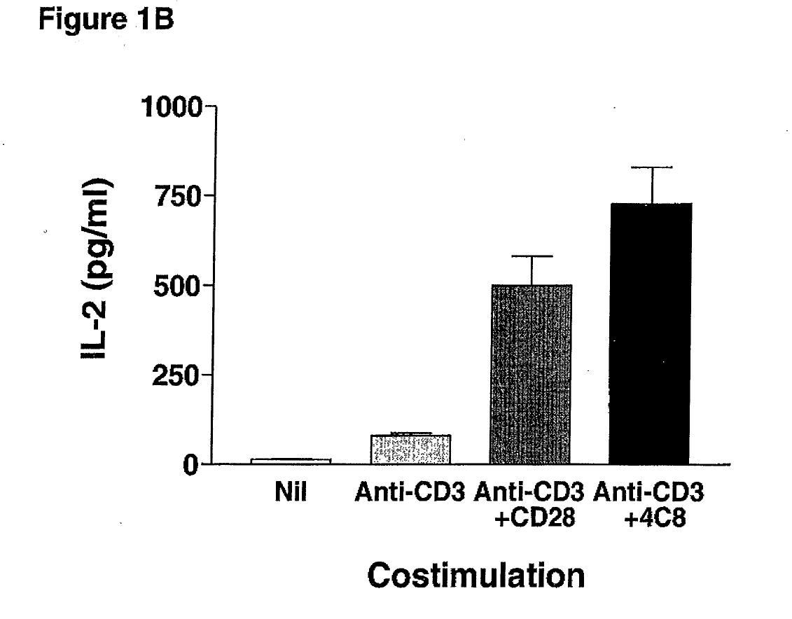

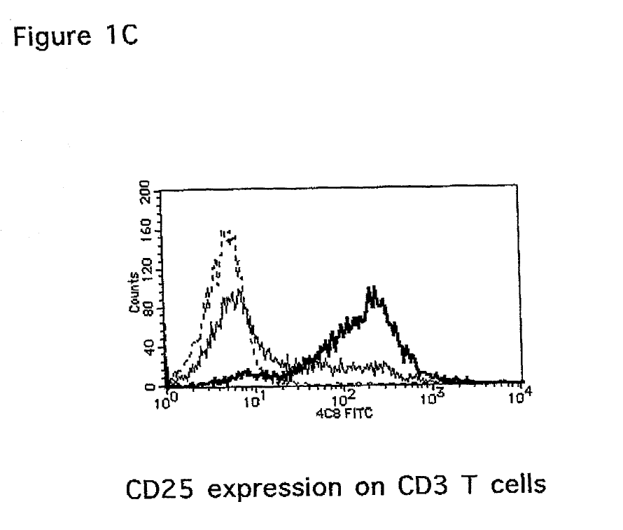

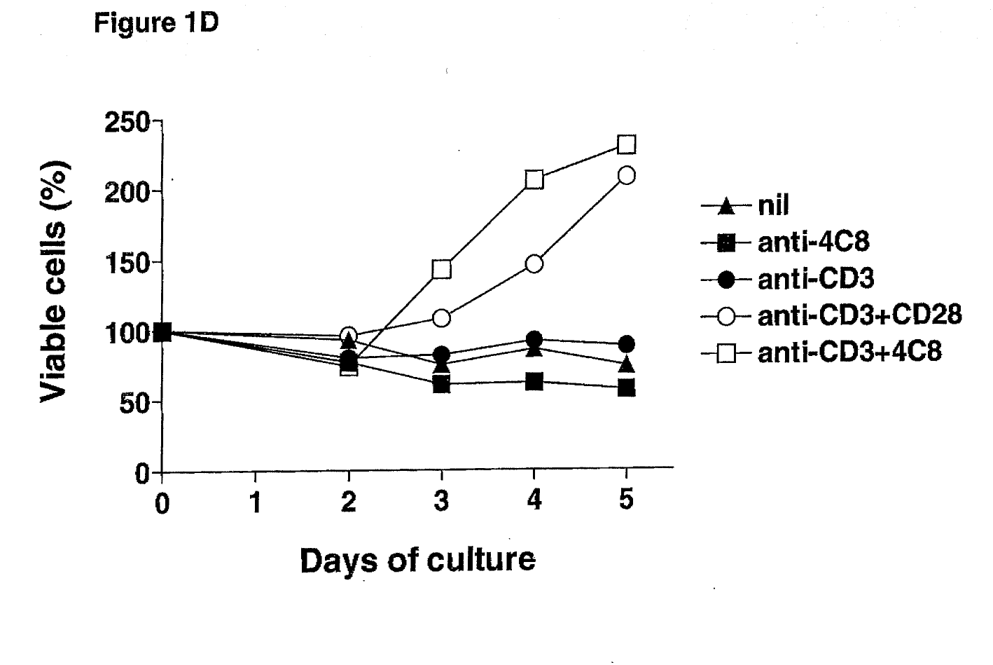

The result is shown in FIG. 1A. Unlike the presence of anti-CD3 mAb alone, coexistence of anti-CD3 and anti-4C8 mAbs greatly enhanced ³H-thymidine incorporation ability of T cells. The presence of anti-4C8 mAb alone had no effect, suggesting that anti-4C8 mAb delivers costimulatory signals to T cells. Enhanced incorporation of ³H-thymidine was also observed for the control group stimulated with anti-CD3 mAb and anti-CD28 mAb. However, under the experimental conditions, anti-4C8 mAb had stronger costimulation ability compared to anti-CD28 mAb. Moreover, anti-4C8 mAb was shown to act on both naive T cells (CD3⁺CD45RA⁺ cells) and memory T cells (CD3⁺, CD45RO⁺ cells), and memory T cells were shown to have higher proliferative ability for both anti-4C8 mAb and anti-CD28 mAb, compared to naive T cells.

Costimulation mediated by molecules other than CD28 which are constantly expressed on T cells, such as CD2, CD9 and CD11a, also enhances incorporation of ³H-thymidine by T cells to a degree equivalent to that of stimulation with CD28. However, unlike the stimulation through CD28, the costimulation is known to be unable to maintain proliferation of T cells because of its low ability of inducing IL-2 production, and to cause apoptosis of the activated T cells. Accordingly, 24 hours after stimulation under conditions similar to those for the experiment of incorporation of ³H-thymidine, IL-2 concentration in the culture supernatant was measured using an ELISA kit (Biosource) using CD3⁺ T cells (FIG. 1B). Production of IL-2 in large quantity was induced by 4C8 costimulation or by CD28 costimulation: the former stimulation induced enhanced IL-2 production of about 50% or more compared to the latter. However, in both cases, IL-2 in the culture supernatant rapidly decreased with time of culturing. On day 3 of culturing, IL-2 decreased below the detection limit. For T cells subjected to costimulation to maintain continuous proliferation, CD25, which is an IL-2 receptor, should be highly expressed. Thus, T cells following 4C8 costimulation and T cells following CD28 costimulation, which were then cultured for 24 hours, were compared for expression of CD25. Expression of CD25 was analyzed by FACScan (Becton-Dickinson, Mountain View, Calif.) after incubating 2-3×10⁵ cells with FITC-labeled anti-CD25 mAb (clone M-A261, PharMingen, San Diego, Calif.) in the presence of 0.05% sodium azide for 20 min in ice, washing twice with 1 ml of PBS/0.1% BSA/0.05% sodium azide, followed by fixing with 0.5 ml PBS/1% paraformaldehyde. As shown in FIG. 1C, 4C8 costimulation resulted in a significantly higher proportion of CD25-expressing T cells compared to CD28 costimulation (4C8:98%, CD28:25%).

4C8 costimulation induced a rapid increase in CD25-expressing cells, such that 75% of cells expressed CD25 at 24 hours after culturing, while CD28 costimulation merely caused a gradual increase in the number of CD25⁺ cells with time of culturing.

4C8 and CD28 costimulation were compared for viable cell count on days 2 to 5 after culturing, taking the cell count before culturing as 100% (FIG. 1D). After culturing under the above stimulation conditions, the cells were collected from each group/3 wells of a 96-well microtiter plate, and then the number of viable cells were counted by a trypan blue dye exclusion method. With CD28 costimulation, cell count started to gradually increase on day 3 after culturing, and became double on day 5 after culturing. With 4C8 costimulation, a similar increase in cell count was seen, however, on day 2 after culturing, a transient, slight decrease, followed by a rapid increase in cell count was observed. Thus, 4C8 costimulation induced a more increase in cell count than that induced by CD28 costimulation. In contrast, stimulation with anti-CD3 mAb alone or with anti-4C8 mAb alone caused no increase in cell count, and cell count started to decrease on day 5 after culturing.

Induction of BCl-x_{L}, which is an anti-apoptotic gene product, suppresses apoptosis induced by stimulation from a T cell antigen receptor in CD28 costimulation, and thus plays an important role in increasing T cell count. Actually, costimulation with those other than CD28 induces no increase in T cell count, no BC1-x_{L}, and no production of 1L-2. Therefore, whether or not Bcl-x_{L} is induced by 4C8 costimulation which induces increased cell count was verified by western blotting. T cells activated by the above method were stimulated. At 24 hours after stimulation, 5×10⁵ viable cells were collected and washed with PBS, and then lysed in a cytolytic buffer (1% Nonidet P-40, 0.1% SDS, 1% sodium deoxycholate, 30 μg/ml aprotinin, 50 μg/ml leupeptin, 100 μg/ml PMSF, 1 mM sodium orthovanadate). Subsequently, the solution was boiled with a SDS sample buffer, subjected to 12% SDS-PAGE, and then transferred from the gel to a nitrocellulose membrane. The nitrocellulose membrane was blocked with a 5% skim milk-containing Tris buffer (10 mM Tris-HCL, pH 7.5, 150 mM NaCl, 0.05% Tween 20), and then allowed to react in 1% skim milk-containing Tris buffer with anti-Bcl-x_{L} polyclonal antibodies (Santa Cruz Biotechnology). After washing the nitrocellulose membrane, horseradish peroxidase-labeled secondary antibodies (1/10,000 dilution) were added for reaction, and developed with an ECL chemiluminescence reagent (Amersham). On day 3 after culturing, similarly to CD28 costimulation, expression of BC1-x_{L} was also confirmed for 4C8 costimulation.

The above results suggest that co-stimulation of anti-4C8 mAb via 4C8 antigen with CD3 induces T cells to proliferate in association with induction of IL-2 production, induction of CD25 expression, and induction of BC1-x_{L} expression. The 4C8 antigen is a costimulatory molecule which induces maximum activation of T cells.

### Example 2

### Pattern of Cytokine Production From CD4⁺T Cells Induced by 4C8 Costimulation

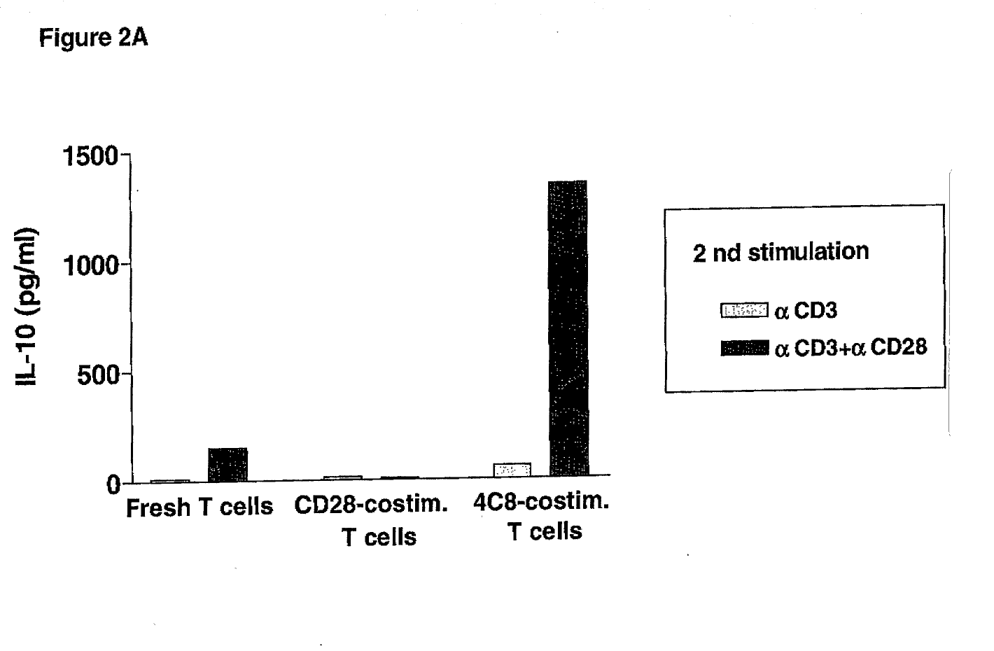

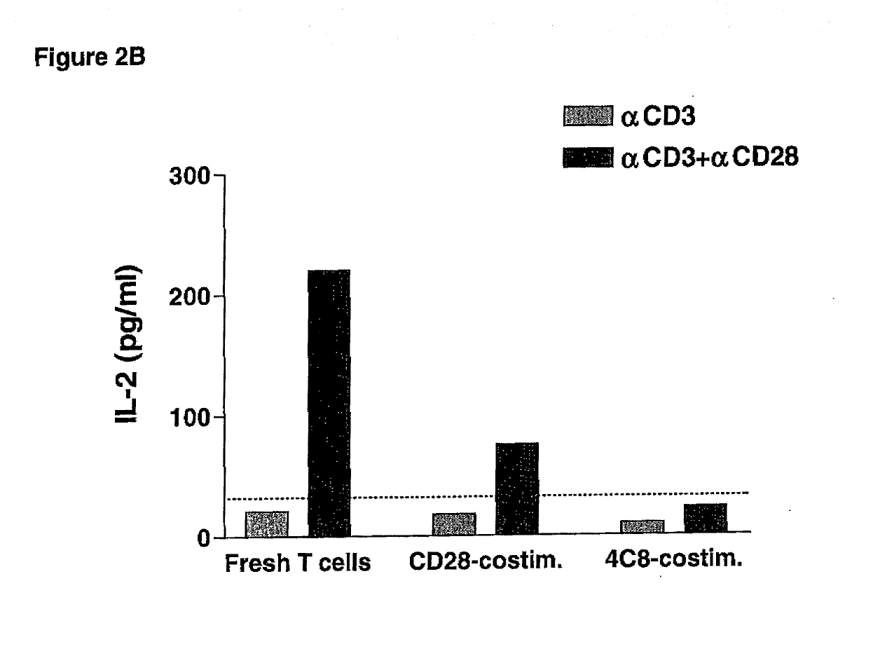

It is known that when naive T cells activated under certain conditions (primary stimulation) are stimulated again (secondary stimulation), differentiated T cells, called Th1 cells or Th2 cells having polarized cytokine production patterns are induced (polarization of Th cells). Whether 4C8 costimulation induces such polarization of T cells was examined using production of IL-2 and IL-10 as an index. Preparation of T cells and primary stimulation of T cells including 4C8 costimulation and CD28 costimulation were performed in the same manner as described in Example 1. On day 3 after culturing for primary stimulation, the cells were collected and washed. The cells were allowed to be in resting state by culturing under no-stimulation for 4 to 6 days, and then secondary stimulation was performed by the method shown in FIG. 2. Specifically, in secondary stimulation, 0.1 μg/ml anti-CD3 mAb was immobilized on a flat bottom 96-well plate, to which 1×10⁵ cells were then added. The cells were cultured with or without 5 μg/ml anti-CD28 mAb. The culture medium was collected and measured using an ELISA kit (Biosource) for IL-2 at 24 hours after culturing and for IL-10 at 48 hours after culturing. As a control group upon secondary stimulation, freshly prepared T cells without no stimulation were used. FIG. 2A shows that T cells which had undergone 4C8 costimulation produced a large amount of IL-10 following secondary stimulation with anti-CD3 mAb and anti-CD28 mAb. In contrast, freshly prepared T cells and T cells which had undergone CD28 costimulation produced a small amount of IL-10 and trace amount of IL-10, respectively following stimulation with anti-CD3 mAb and anti-CD28 mAb. Further, T cells undergone 4C8 costimulation produced IL-2 at a level below the detection limit following secondary stimulation with anti-CD3 mAb and anti-CD28 mAb. In contrast, freshly prepared T cells and T cells which had undergone CD28 costimulation significantly produced IL-2, wherein the latter produced IL-2 at a level lower than that of the former (FIG. 2B). No group was induced to produce cytokines following stimulation with anti-CD3 mAb alone. T cells induced by 4C8 costimulation clearly differed from T cells induced by CD28 costimulation in cytokine production pattern. It is suggested from the results that T cells induced by 4C8 costimulation are similar to regulatory T cells in that these cells produced mainly a large amount of IL-10 but produced no IL-2.

### Example 3

### CD4⁺ T Cells Induced by 4C8 Costimulation are Hypo-Anergic to Anti-CD3 Stimulation

Regulatory T cells are known to be hypo-anergic or anergic to polyclonal or antigen-specific stimulation. Thus the proliferative ability of T cells which had been induced by 4C8 costimulation was examined upon stimulation with anti D3 mAb. Cells induced in a manner similar to that in Example 1 by 4C8 costimulation and CD28 costimulation were cultured. On day 3 after culturing, the cells were collected, washed, and then cultured for 4 to 6 days without stimulation to allow being resting state. Then, 1×10⁵ cells/well (200 μl/well) were plated onto a U-shaped bottom 96 well microtiter plate, and then soluble anti-CD3 mAb (10 ng/ml) was added thereto in the presence of irradiated (6,000 rads) peripheral blood mononuclear cells (4×10⁵ cells/well). Then, the cells were cultured for 3 days in the presence or absence of anti-CD28 mAb (5 μg/ml) or IL-2 (100 U/ml), so that incorporation of ³H-thymidine was measured in the same manner as in Example 1. At this time, freshly prepared T cells were used similarly to Example 2.

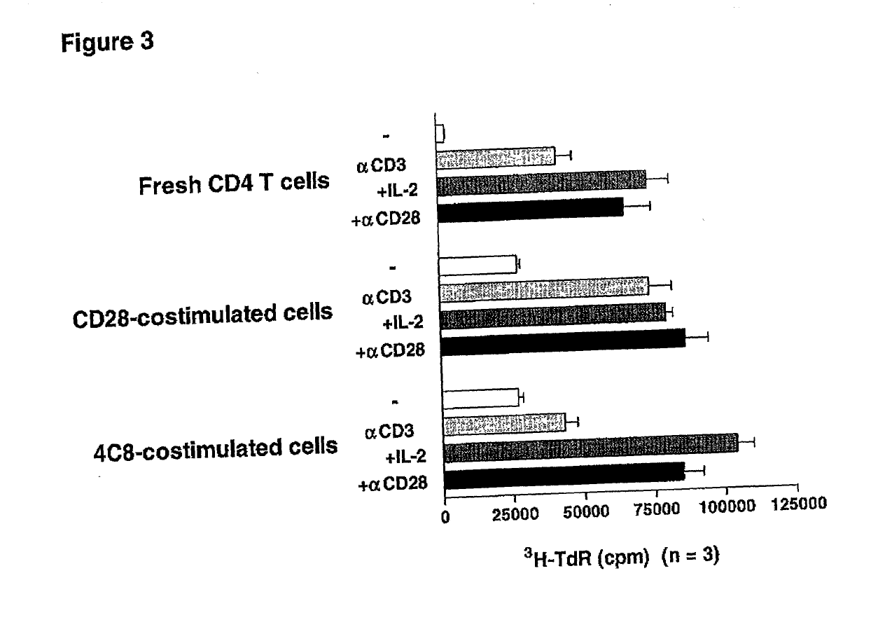

As shown in FIG. 3, both the freshly prepared T cells and T cells induced by CD28 costimulation showed strong proliferative reaction following stimulation with anti-CD3 mAb. The proliferative reaction was further enhanced by additional stimulation with IL-2 or with anti-CD28 mAb. In contrast, T cells induced by 4C8 costimulation showed slight proliferative reaction following stimulation with anti-CD3 mAb, but the proliferative reaction was greatly enhanced by additional stimulation with IL-2 or with anti-CD28 mAb. In particular, additional stimulation with IL-2 resulted in constant high reactivity compared to freshly prepared T cells and T cells induced by CD28 costimulation. These results suggest that T cells induced by 4C8 costimulation are hyporesponsive to stimulation with anti-CD3 mAb, similar to regulatory T cells, but the hyporesponsiveness is canceled by the addition of IL-2 or anti-CD28 mAb.

### Example 4

### CD4⁺ T Cells Induced by 4C8 Costimulation Inhibit Activation of Other T Cells by polyclonal Stimulation

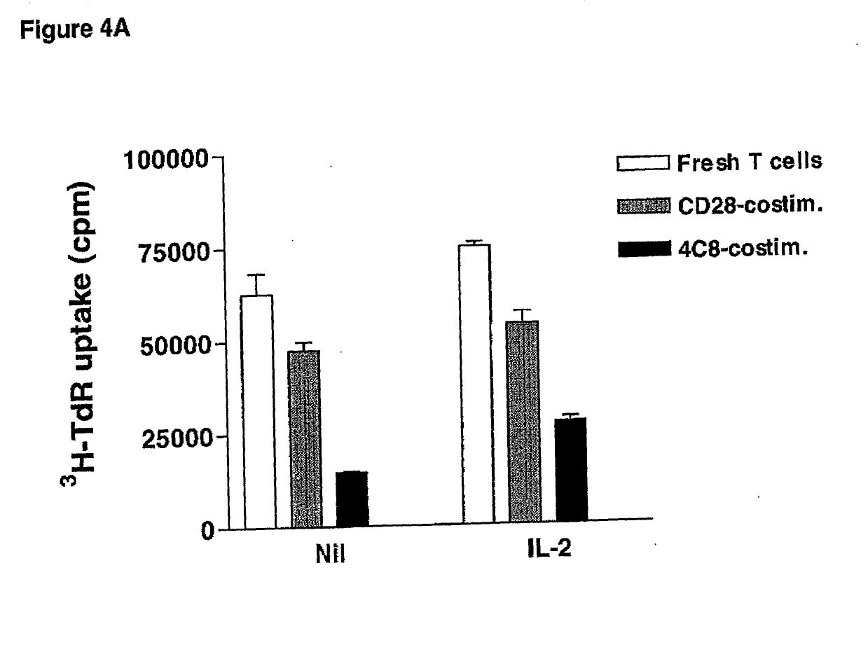

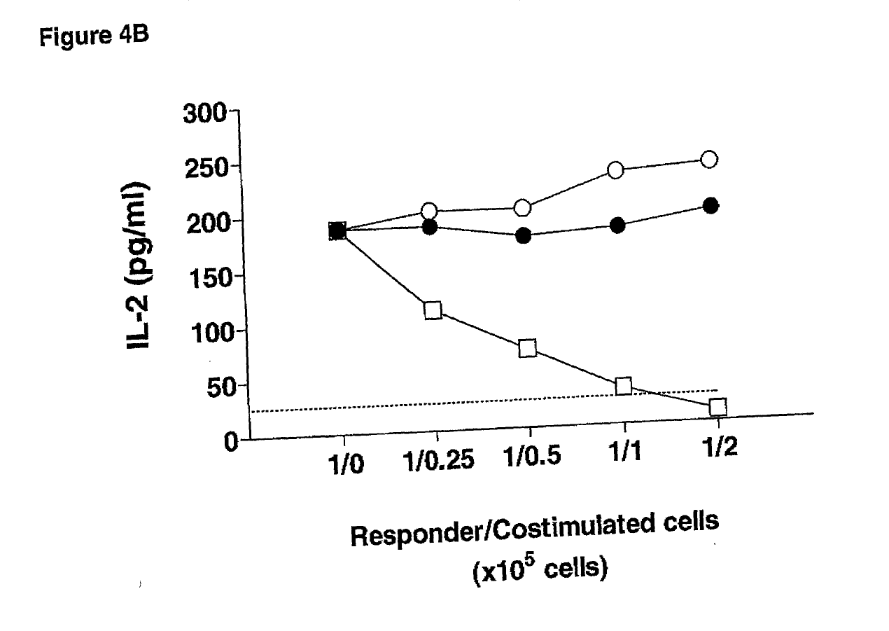

Regulatory T cells are known to inhibit proliferative reaction of other T cells. Therefore, inhibitory ability of T cells induced by 4C8 costimulation was examined. In a manner similar to Example 3, T cells which had undergone 4C8 costimulation, T cells which had undergone CD28 costimulation, and freshly prepared T cells (controls) were irradiated at 6,000 rads. Next, these T cells were respectively mixed with freshly prepared CD4⁺ T cells (1×10⁵ cells/well) at the cell count ratio of 1:1. Soluble anti-CD3 mAb (10 ng/ml) was added in the presence of irradiated (6,000 rads) peripheral blood mononuclear cells (4×10⁵ cells/well). The cells were cultured for 3 days, and then ³H-thymidine uptake was measured. The cells used here in combination were derived from the same donor. As shown in FIG. 4A, the group co-cultured with the cells following 4C8 costimulation showed approximately 80% proliferative inhibition compared to that of the control group co-cultured with freshly prepared T cells. In contrast, the group co-cultured with the cells following CD28 costimulation showed only about 25% inhibition. When IL-2 (100 U/ml) was added to the assay system, the group co-cultured with the cells following 4C8 costimulation showed proliferative reaction of about twice as much as that in the absence of IL-2, but unlike the control group, the inhibitory effect was not canceled. The inhibitory action of regulatory T cells has been reported to inhibit production of IL-2 by other T cells. Therefore, in a system similar to the above system, IL-2 in the culture supernatant after 24 hours of co-culturing was measured by ELISA with the ratios of cell numbers added varied as shown in FIG. 4B.

With an increased number of cells added, T cells (∘) following CD28 costimulation showed a slight tendency to enhance production of IL-2, compared to the control group (●) to which freshly prepared T cells were added. However, at any cell ratio, almost no difference was observed between T cells (∘) following CD28 costimulation and the control group. However, T cells (□) following 4C8 costimulation inhibited production of IL-2 in a manner dependent on cell number added. Addition of twice the number of cells for reaction resulted in the production of IL-2 below the detection limit. These results suggest that unlike CD28 costimulation, 4C8 costimulation induces regulatory CD4⁺ T cells capable of inhibiting the proliferative reaction of other T cells by inhibiting IL-2 production.

### Example 5

### CD4⁺ T cells induced by 4C8 costimulation maintain high expression of CD25 on cell surface and CD152 intracellularly)

CD4⁺ regulatory T cells highly express CD25 on the cell surfaces and CD152 (CTLA-4) intracellularly. Therefore, the proportion of cells expressing CD25 and CD152 (PE labeled anti-CD152 mAb (Sigma Chemical Co.)) on the cell surfaces was analyzed by FACScan (in a manner similar to that in Example 1) when the cells on day 3 following 4C8 costimulation and CD28 costimulation were collected, washed and then cultured for another 3 days or 6 days without stimulation.

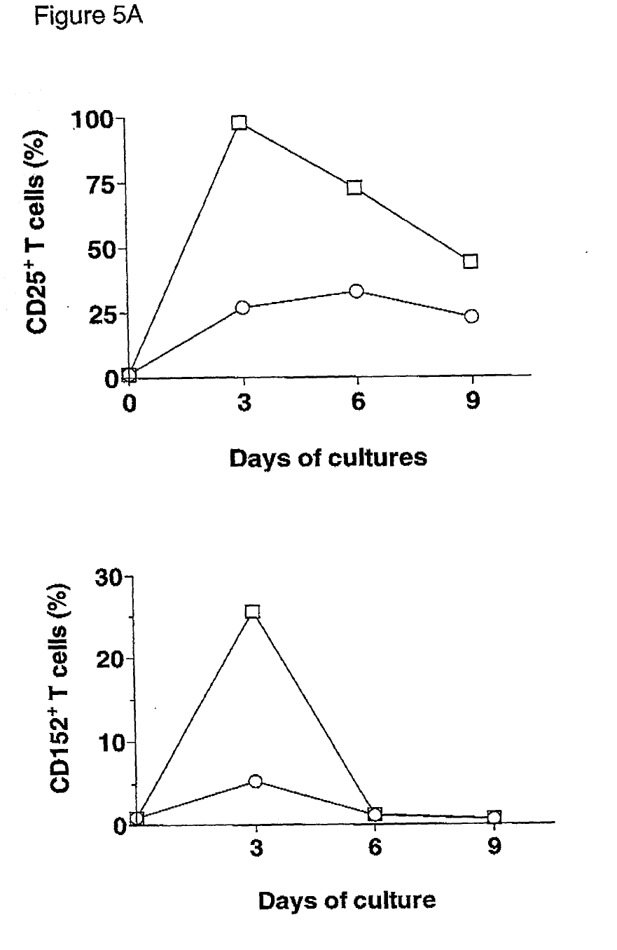

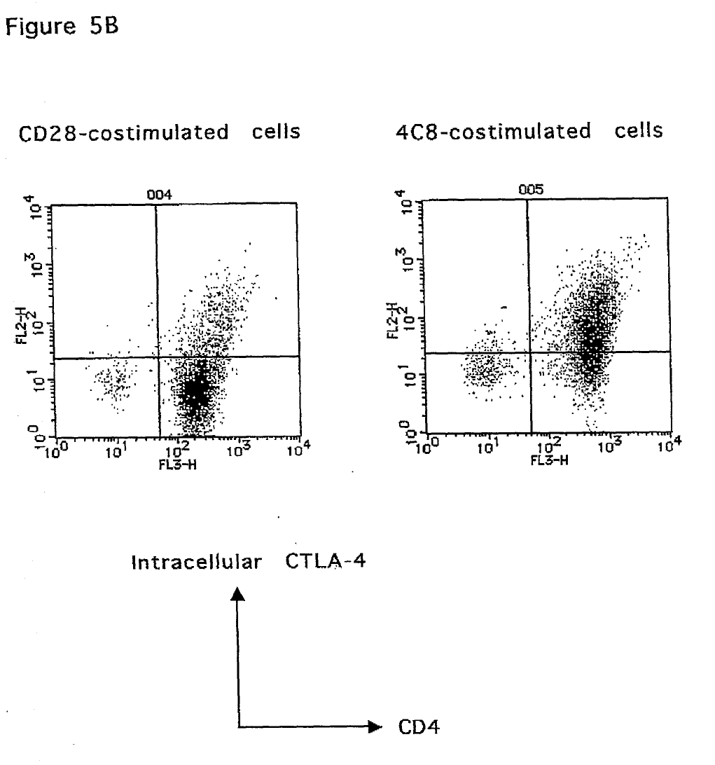

A high proportion of the cells following 4C8 costimulation (□) maintained expression of CD25 even in a culturing period in resting state after stimulation, compared to the cells following CD28 costimulation (∘) (Top panel in FIG. 5A). In contrast, the proportion of the cells expressing CD152 on the cell surfaces was greatly higher for the cells on day 3 following 4C8 costimulation than that of the cells following CD28 costimulation; and the proportion dropped to the background level on day 3 after culturing in resting state after stimulation for both the cells following 4C8 costimulation and the cells following CD28 costimulation (Bottom panel in FIG. 5A). Intracellular expression of CD152 was analyzed for the cells on day 4 after culturing in resting state after costimulation that had been observed no expression on the cell surfaces. Specifically, the cells were first stained with Quantum Red™-labeled anti-CD4 mAb (Sigma Chemical Co.), fixed with PermeaFix (Ortho Diagnostics, Raritan, N.J.), subjected to permeation, and then stained with PE-labeled anti-CD152 mAb (Sigma Chemical Co.). While most cells following 4C8 costimulation expressed CD152 intracellularly (Right panel in FIG. 5B), the expression was observed in only a slight number of cell populations among the cells following CD28 costimulation (Left panel in FIG. 5B).

### Example of Indications

T cells induced by 4C8 costimulation from human peripheral blood T cells highly expressing CD25 and CD152, and highly producing IL-10, are capable of inhibiting proliferative response of other T cells, and have properties similar to those of suppressor CD4⁺ regulatory T cells that have been reported so far in animals and humans. The present invention reveals that 4C8 costimulation induces such T cells to proliferate in association with increased cell number. It is suggested that with another stimulation or the stimulation in combination with cytokines, such as IL-2, regulatory T cells enable ex vivo proliferation of cells in large quantity. Actually in an animal experiment, these cells transferred to animals act suppressively on autoimmune disease, transplant rejection or GvHD (Hara, M. et al., 2001. J. Immunol. 166:3789-3796.; Taylor, P. A. et al., 2001. J. Exp. Med. 193:1311-1317). Hence, cellular medicine using immunosuppressive action of the regulatory T cells can be applied to therapy for autoimmune disease, such as rheumatism, transplantation or the like.

The present invention provides a method for inducing differentiation and promoting proliferation of regulatory T cells, and a pharmaceutical composition to be used in the method. The pharmaceutical composition of the present invention is useful in therapy for autoimmune disease, transplantation or the like based on cellular medicine using the immunosuppressive action of the regulatory T cells.

All publications, patents and patent applications cited herein are incorporated herein by reference in their entirety.

What is claimed is:

1. A method for inducing differentiation and/or promoting proliferation of regulatory T cells, comprising allowing a CD3 agonist and a 4C8 antigen agonist to act on an immunocyte expressing CD3 and 4C8 antigens.

2. The method of claim 1, wherein the immunocyte expressing CD3 and 4C8 antigens is contained in peripheral blood, lymph nodes or thymic gland.

3. The method of claim 1, wherein the immunocyte expressing CD3 and 4C8 antigens is a T cell.

4. The method of claim 1, wherein the immunocyte expressing CD3 and 4C8 antigens is a peripheral blood mononuclear cell.

5. The method of claim 1, wherein the CD3 agonist is an anti-CD3 antibody.

6. The method of claim 1, wherein the 4C8 antigen agonist is an anti-4C8 antibody.

7. The method of claim 6, wherein the anti-4C8 antibody is a humanized antibody or a human antibody.

8. The method of claim 1, wherein the CD3 agonist and 4C8 antigen agonist act in vivo on an immunocyte expressing CD3 and 4C8 antigens.

9. The method of claim 1, wherein the CD3 agonist and 4C8 antigen agonist act ex vivo on an immunocyte expressing CD3 and 4C8 antigens.

10. The method of claim 1, wherein the regulatory T cell induced to differentiate expresses CD25 and CD152 and produces IL-10.

11. The method of claim 1, wherein the regulatory T cell induced to differentiate suppresses cytokine production, proliferation and/or activation of other T cells.

12. A pharmaceutical composition for inducing differentiation and/or promoting proliferation of a regulatory T cell, comprising as an active ingredient a 4C8 antigen agonist.

13. The pharmaceutical composition of claim 12, wherein the 4C8 antigen agonist is an anti-4C8 antibody.

14. The pharmaceutical composition of claim 13, wherein the 4C8 antibody is a humanized antibody or a human antibody.
